(19) United States
(12) Plant Patent Application Publication
(10) Pub. No.: US 2002/0162146 P1
(43) Pub. Date: Oct. 31, 2002

(54) Hybrid tea rose plant named 'Tanavl'

(75) Inventor: Hans-Jurgen Evers, Uetersen (DE)

Correspondence Address: KLARQUIST SPARKMAN CAMPBELL, LEIGH & WHINSTON, LLP, One World Trade Center, Suite 1600, 121 S.W. Salmon Street, Portland, OR, 97204, US

(73) Assignee: Rosen Tantau Mathias Tantau Nachfolger

(21) Appl. No.: 09/846,145

(22) Filed: Apr. 30, 2001

(51) Int. Cl. A01H 5/00

(52) U.S. Cl. PLT/132

(57) ABSTRACT

Hybrid tea rose plant having flowers of extrordinary copper-yellow color.

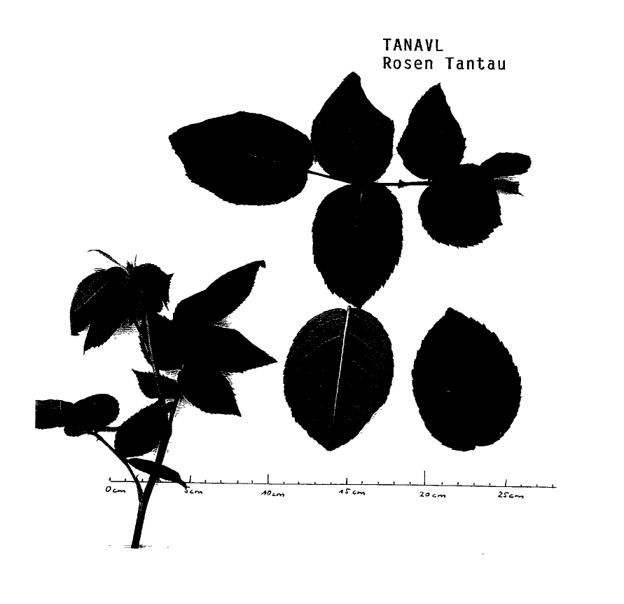

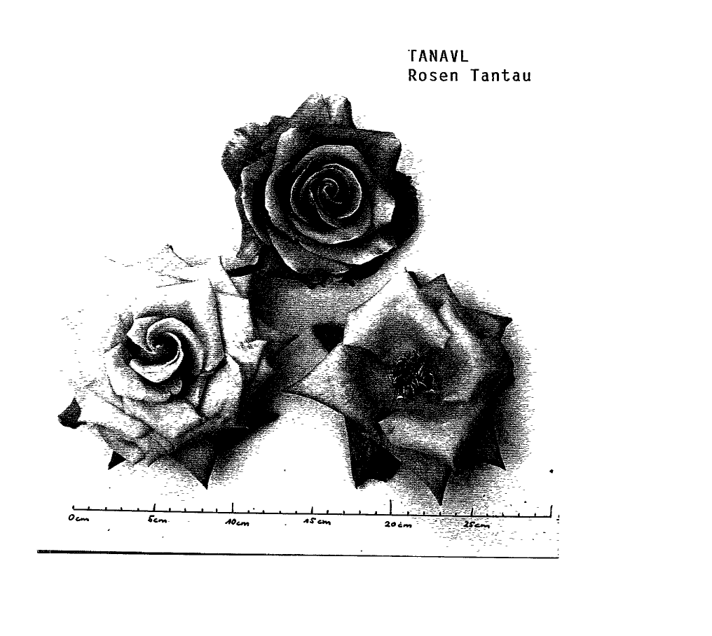

The present invention relates to a new and distinct variety of rose plant of the hybrid tea class which was originated by me by crossing two unnamed varieties.

The primary objective of this breeding was to produce a new rose variety having vigorous growth, high production of cut flowers of an extraordinary copper-yellow color. This objective was substantially achieved.

Asexual reproduction of this new variety by budding onto Rosa canina rootstock, as performed at Uetersen, Germany, shows that the foregoing and all other characteristics and distinctions come true to form and are established and transmitted through succeeding propagations.

The accompanying illustration shows typical specimens of the vegetative growth and flowers of this new variety in different stages of development, depicted in color as nearly true as it is reasonably possible to make the same in a color illustration of this character.

The following is a detailed description of my new rose cultivar with color descriptions using terminology in accordance with The Royal Horticultural Society (London) colour chart, except where ordinary dictionary significance of color is indicated.

Parentage:

Seed Parent.—Unnamed.

Pollen Parent.—Unnamed.

Classification:

Botanical.—Rosa 1.

Commercial.—Hybrid tea.

### FLOWER Observations made from specimens grown in a greenhouse environment at Uetersen, Germany.

Blooming Habit: Continuous.

Bud:

Size.—About 6 cm long when the petals start to unfurl.

Form.—The bud form is long.

Color.—When sepals first divide, bud color is Orange Group 25B. When half blown, the upper sides of the petals are Orange Group 24A; and the lower sides of the petals are Orange Group 24A.

Sepals.—Color: Green Group 142A. Surface texture: Smooth. There are three normally appendaged sepals. There are five unappendaged sepals with hairy edges.

Receptacle.—Color: Green Group 143C with White Group 155A. Shape: Apple. Size: Medium, about 1.5 cm. Surface: Mossy.

Peduncle.—Length: Long, about 8 cm. Surface: Smooth. Color: Green Group 143A. Strength: Erect.

Bloom:

Size.—Large. Average open diameter is 13 cm.

Borne.—Singly.

Stems.—Strength: Strong. Average length is about 80 cm.

Form.—When first open: Globular. Permanence: Outer petals curl back.

Petalage.—Number of petals under normal conditions: 35.

Color.—The upper sides of the petals are Orange Group 24A on outer portions shading to Orange Group 25A on inner portions. The reverse sides of the petals are Yellow-Orange Group 21A on outer portions and shading to Yellow-Orange Group 21 C on inner portions. The base of each petal has a Yellow-Ornage Group 16A spot. The major color on the upper side is Orange Group 24A.

Variegations.—Orange Group 24A to Yellow-Orange Group 23B.

Discoloration.—The general tonality at the end of the first day: Yellow-Orange Group 23A. At the end of the third day: Orange Group 24B. As bloom ages further color fades to Yellow-Orange Group 22C.

Fragrance.—Slight. Sweetly spicy.

Petals:

Texture.—Leathery.

Shape.—Round.

Form.—Tips slightly recurved and edges slightly quilled.

Arrangement.—Irregular. Petaloids in the center are few.

Persistence.—Petals drop off cleanly before drying.

Lastingness.—On the plant: Long (about 17 days). As a cut flower: Long (about 12 days).

Reproductive Parts:

Anthers.—Size: Medium. Quantity: Many. Color: Bright yellow. Arrangement: Regular around styles.

Filaments.—Color: Orange Group 28A to Yellow-Orange Group 15A.

Pollen.—Color: Yellow-Orange Group 15A.

Styles.—Color: Yellow-Orange Group 23D.

Stigmas.—Color:

Plant:

Form.—Pillar.

Growth.—Vigorous. At maturity, height is 100 cm, width is 30 cm.

Foliage: Number of leaflets on normal mid-stem leaves: 5.

Size.—Large (11 cm long ×7 cm wide).

Quantity.—Normal.

Color.—New foliage: Upper surface: Green Group 139A. Lower surface: Green Group 138A. Old foliage: Upper surface: Green Group 139A. Lower surface: Green Group 138A.

Leaflets:

Shape.—Oval.

Texture.—Leathery.

Edge.—Serrated.

Serration.—Single.

Petiole Rachis.—Color: Green Group 138B.

Petiole Underside.—With prickles.

Stipules.—Medium, serrated.

Disease Resistance.—Resistant to blackspot and rust with average resistance to mildew as a cut rose variety grown in a greenhouse.

Wood:

New Wood.—Color: Green Group 139D. Bark: Smooth.

Old Wood.—Color: Yellow-Green Group 152A. Bark: Rough.

Prickles:

Quantity.—On main canes from base: Ordinary.

Form.—Long, hooked downward.

Color when Young.—Green Group 137D.

Color when Mature.—Greyed-Red Group 178B.

Small Prickles:

Quantity.—On main stalks: Few.

Color.—Green Group 137D.

I claim:

1. A new and distinct variety of rose plant of the hybrid tea class, substantially as herein shown and described, characterized particularly by the extraordinary copper-yellow color of its flowers.
